(19) United States
(12) Patent Application Publication
(10) Pub. No.: US 2016/0256474 A1
(43) Pub. Date: Sep. 8, 2016

(54) TOPICAL SPRAY COMPOSITION OF HALOBETASOL

(71) Applicant: SUN PHARMACEUTICAL INDUSTRIES LIMITED, Mumbai, Maharashtra (IN)

(72) Inventors: Anil RANA, Gurgaon (IN); Sumit MADAN, New Delhi (IN); Anupam TREHAN, Gurgaon (IN); Vinod Kumar ARORA, Gurgaon (IN)

(21) Appl. No.: 15/024,977

(22) PCT Filed: Sep. 22, 2014
(86) PCT No.: PCT/IB2014/064745

§ 371 Date: Mar. 25, 2016

(30) Foreign Application Priority Data
Sep. 25, 2013 (IN) 2839/DEL/2013

(51) Int. Cl. A61K 31/573; A61K 9/12; A61K 47/12; A61K 47/14; A61K 47/06; A61K 9/00; A61K 47/10

(52) U.S. Cl. CPC A61K 31/573; A61K 9/0014; A61K 9/124; A61K 47/10; A61K 47/14; A61K 47/06; A61K 47/12

(57) ABSTRACT

The present invention relates to topical spray compositions comprising halobetasol, an emollient, a non-aqueous solvent, and a propellant. It also relates to a process for the preparation of the topical spray compositions. It further relates to a method of treating topical skin condition by administering said topical spray compositions.

### FIELD OF THE INVENTION

The present invention relates to topical spray compositions comprising halobetasol, an emollient, a non-aqueous solvent, and a propellant; a process for their preparation; and a method of treating a topical skin condition by administering said topical spray compositions.

### BACKGROUND OF THE INVENTION

Many delivery systems, including creams, ointments, lotions, gels, transdermal patches, and sprays, are currently available for the topical application of active ingredients.

Amongst these, semisolid dosage forms such as creams, ointments, lotions, and gels are widely used. However, these are often subject to unintended removal or transfer to other skin surfaces after being applied on the skin. In addition, when a semisolid dosage form is applied on skin, it is typically “rubbed in” which may further irritate the intended site of application. These dosage forms may also cause clogging of pores and therefore block delivery of a suitable quantity of the active ingredient to the skin.

Transdermal patches have fixed shapes and sizes and work best on skin areas that are relatively flat and that do not flex or stretch. However, these comprise an occlusive backing membrane which often results in local skin irritation.

Pharmaceutical sprays exhibit numerous advantages over other known topical delivery systems. These advantages include the ease with which the formulation can be delivered to the areas of the body that are difficult to treat, the possibility of controlling the dose, and the absence of contamination during use. Further, sprays are more suitable when application is required for a large area of skin and therefore result in enhanced patient compliance.

U.S. Pat. Nos. 6,126,920 and 7,078,058 disclose betamethasone valerate foamable spray compositions comprising a quick-break foaming agent, an aliphatic alcohol, a fatty alcohol, a surface active agent, buffering agent, water, and a propellant.

U.S. Pat. No. 7,645,803 discloses a foamable spray composition comprising a saccharide, a surface active agent, a polymeric agent, a gelling agent, a film-forming agent, water, and a propellant.

U.S. Publication No. 2008/0206155 discloses a non-alcoholic foaming pharmaceutical emulsion composition comprising a steroid, an unctuous emollient, and at least one liquefied or compressed gas propellant.

U.S. Publication No. 2008/0107758 discloses a topical spray composition comprising a corticosteroid, an alcohol, a propellant, and a blend of three or more botanic seed oils that are prepared by a cold press method.

U.S. Pat. No. 6,579,512 discloses a topical spray composition comprising clobetasol, an alcohol, isopropyl myristate, and a propellant.

Halobetasol is a high potency corticosteroid. Topical dosage forms of halobetasol, such as creams and ointments, are commercially available under the trade name Ultravate® and have been used for the relief of inflammatory and pruritic manifestations of corticosteroid-responsive dermatoses.

Although cream and ointment dosage forms of halobetasol have been known for decades, there remains an unmet need for an improved topical composition of halobetasol which overcomes the drawbacks of the available cream and ointment dosage forms and results in better patient compliance.

The present invention teaches topical spray compositions of halobetasol which are non-occlusive, non-irritant, and provide enhanced patient compliance.

### SUMMARY OF THE INVENTION

The compositions of the present invention are a significant advance over conventional halobetasol compositions, since they allow for the application of halobetasol with no physical contact to the area of application, except by the spray itself.

The present invention relates to non-occlusive, non-irritant, quick drying topical spray compositions of halobetasol. The present invention includes a topical spray composition of halobetasol comprising halobetasol, an emollient, a non-aqueous solvent, and a propellant. It also relates to a process for the preparation of said topical spray composition. It further relates to a method of treating topical skin conditions by administering said topical spray composition.

### DETAILED DESCRIPTION OF THE INVENTION

A first aspect of the present invention provides a topical spray composition of halobetasol comprising halobetasol, an emollient, a non-aqueous solvent, and a propellant.

According to one embodiment of this aspect, there is provided a topical spray composition of halobetasol comprising halobetasol, an emollient, a non-aqueous solvent, and a propellant, wherein the emollient is selected from the group consisting of fatty acid triglycerides, fatty acid esters, and polyhydric alcohols.

According to another embodiment of this aspect, there is provided a topical spray composition of halobetasol comprising halobetasol, an emollient, a non-aqueous solvent, and a propellant, wherein the composition is stable.

A second aspect of the present invention provides a dispensing system for administering a topical spray composition of halobetasol comprising halobetasol, an emollient, a non-aqueous solvent, and a propellant, wherein the dispensing system comprises a container and a valve assembly.

A third aspect of the present invention provides a process for the preparation of a topical spray composition of halobetasol comprising:
(a) dissolving halobetasol in one portion of a non-aqueous solvent to form a solution;
(b) mixing an emollient and another portion of the non-aqueous solvent into the solution of step (a);
(c) dispensing the solution of step (b) in a dispensing system; and
(d) charging a propellant in the dispensing system of step (c).

A fourth aspect of the present invention provides a method of treating a topical skin condition by administering a topical spray composition of halobetasol comprising halobetasol, an emollient, a non-aqueous solvent, and a propellant.

According to one embodiment of this aspect, there is provided a method of treating a topical skin condition by administering a topical spray composition of halobetasol comprising halobetasol, an emollient, a non-aqueous solvent, and a propellant, wherein the condition is selected from the group consisting of dermatoses, psoriasis, eczema, rosacea, acne vulgaris, dermatitis, pruritus, seborrhea, skin cancers, inflammation, and combinations thereof.

According to another embodiment of this aspect, there is provided a method of treating a topical skin condition by administering a topical spray composition of halobetasol comprising halobetasol, an emollient, a non-aqueous solvent, and a propellant, wherein the method comprises co-administration of at least one additional drug used to treat topical skin conditions.

The term “topical”, as used herein, refers to a composition meant for application to the skin, nail, or mucosal tissue.

The term “spray”, as used herein, means to dispense the composition as a mass or jet of droplets from a dispensing system.

The term “stable”, as used herein, means chemical stability wherein not more than 5% w/w of total related substances are formed on storage at 40° C. and 75% relative humidity or at 25° C. and 60% relative humidity for a period of at least three months to the extent necessary for sale and use of composition.

The term “halobetasol”, as used herein, includes halobetasol and its salts, polymorphs, hydrates, solvates, prodrugs, chelates, and complexes. The preferred salt of halobetasol is halobetasol propionate. The topical spray composition of the present invention comprises halobetasol in an amount from about 0.01% w/w to about 0.5% w/w of the total composition.

The term “emollient”, as used herein, refers to a substance that helps retain skin moisture and also helps control the rate of evaporation and the tackiness of the composition. Additionally, emollients provide a softening or soothing effect on the skin surface. Suitable examples of emollients are selected from the group consisting of fatty acid triglycerides such as mixtures of caprylic and capric triglycerides (e.g., Crodamol™ GTCC-LQ, Miglyol®, Captex®, Labrafac™ Lipophile WL), palmitic triglyceride, oleic triglyceride, caprylic triglyceride, capric triglyceride, and linoleic triglyceride; fatty acid esters such as isopropyl myristate, isopropyl palmitate, dibutyl adipate, and dibutyl phthalate; polyhydric alcohols such as propylene glycol, butylene glycol, polyethylene glycol, glycerol, and sorbitol; fatty acids such as oleic acid and stearic acid; oils such as mineral oil, lanolin oil, coconut oil, cocoa butter, olive oil, jojoba oil, and castor oil; cyclomethicone; hydrogenated lanolin; waxes; lecithin; or mixtures thereof Preferably, the emollient is selected from the group consisting of fatty acid triglycerides, fatty acid esters, and polyhydric alcohols.

The term “non-aqueous solvent”, as used herein, refers to the solvent used to dissolve halobetasol. Suitable non-aqueous solvents are selected from the group consisting of ethyl alcohol, isopropyl alcohol, propylene glycol, butanediol, pentanediol, hexanediol, triethylene glycol, tetraethylene glycol, dipropylene glycol, dibutylene glycol, glycerin, dimethyl isosorbide, tetrahydro furfuryl alcohol polyethylene glycol ether, N-methyl-2-pyrrolidone, 1-methyl-2-pyrrolidinone, dimethyl sulfoxide, dimethyl acetamide, lactic acid, glycolic acid, methylene chloride, methyl-ethyl-ketone, ethyl acetate, methylene dimethyl ether, and mixtures thereof. In particular, ethyl alcohol is dehydrated ethyl alcohol.

The term “propellant”, as used herein, refers to the substance that helps in propelling the composition out of the container. Suitable examples of propellants are selected from the group consisting of conventional, non-ozone depleting hydrocarbon propellants, including propane, butane, isobutane, cyclopropane, 1,1,1,2-tetrafluorethane, 1,1,1,2,3,3,3-heptafluoropropane, 1,1-difluoroethane, 1,1,1,3,3,3-hexafluoropropane, and mixtures thereof; fluorocarbon gas; and liquefied petroleum gas.

The term “about”, as used herein, refers to any value which lies within the range defined by a variation of up to ±10% of the value.

The topical spray composition of the present invention further comprises solubilizers, permeation enhancers, film-formers, plasticizers, antioxidants, pH-adjusting agents, or mixtures thereof.

The term “solubilizer” as used herein is a substance that aids in the dissolution or dispersion of halobetasol in the composition. Suitable solubilizers are selected from the group consisting of polyhydric alcohols such as propylene glycol and polyethylene glycol; fatty acids such as oleic acid and stearic acid; non-ionic and ionic surfactants such as polyoxyethyl-sorbitan-fatty acid esters such as polysorbates, ethers of sugars, ethoxylated fatty alcohols, sodium lauryl sulfate, taurocholic acid, lecithin, and Labrasol®; vitamin E; vitamin E TPGS (tocopheryl polyethylene glycol 1000 succinate); or combinations thereof.

The term “permeation enhancer” as used herein is a substance used to enhance the penetration rate of halobetasol through the skin. Suitable permeation enhancers are selected from the group consisting of lipophilic solvents such as dimethyl sulfoxide and dimethyl formamide; non-ionic and ionic surfactants such as polyoxyethyl-sorbitan-fatty acid esters such as polysorbates, ethers of sugars, ethoxylated fatty alcohols, sodium lauryl sulfate, taurocholic acid, lecithin, and Labrasol®; fatty acid esters such as isopropyl myristate and isopropyl palmitate; fatty acids such as oleic acid and stearic acid; polyhydric alcohols such as propylene glycol and polyethylene glycol (e.g., polyethylene glycol 400); Transcutol®; essential oils, e.g., menthol; and combinations thereof.

The term “film-former” as used herein is a substance that forms a stable film on a topical surface when applied. Suitable film-formers are selected from the group consisting of acrylic polymers or copolymers such as methacrylic acid copolymers; cellulose derivatives such as cellulose acetate, hydroxypropyl methyl cellulose, hydroxy ethyl cellulose, methyl cellulose, and ethyl cellulose; polyvinyl acetate; polyvinyl alcohol; povidone; povidone vinyl acetate; and combinations thereof These film-formers can partially dissolve on exposure to moisture from the skin or air, resulting in the formation of a porous film. The porosity can be enhanced by including additional water-soluble additives. The water-soluble additive is preferably propylene glycol, sodium lauryl sulphate, poloxamers, polyoxyl 35 castor oil, polyoxyl 40 hydrogenated castor oil, cetomacrogol, polyethylene glycol, transcutol, or combinations thereof.

The term “plasticizer” as used herein is a substance that aids the composition in forming a flexible, adherent film on the skin. Suitable plasticizers are selected from the group consisting of citric acid esters, dimethyl isosorbide, castor oil, propylene glycol, polyethylene glycol, glycerol, oleic acid, citric acid, phosphate esters, fatty acid esters, glycol derivatives, hydrocarbons and their derivatives, adipic acid, butanediol polyesters, diethyl phthalate, dibutyl phthalate, chlorinated paraffins, and combinations thereof.

Suitable antioxidants are selected from the group consisting of butylated hydroxyl anisole, butylated hydroxy toluene, sodium metabisulfite, ascorbic acid, ascorbyl palmitate, thiourea, acetylcysteine, dithiothreitol, cysteine hydrochloride, propyl gallate, tocopherol, and combinations thereof.

Suitable pH-adjusting agents are selected from the group consisting of pharmaceutically acceptable organic or inorganic acids or bases such as sodium hydroxide, tromethamine, hydrochloric acid, inorganic oxides, inorganic salts of weak acids, and combinations thereof.

In the present invention, the dispensing system comprises a container and a valve assembly.

Containers can be made from materials selected from the group consisting of stainless steel, aluminum, plastic, and glass. The plastic container can be made up of high density polyethylene (HDPE). The containers can be coated with an inert inner lining of epoxy-phenolic resins, epoxy-urea-formaldehyde resins, polytetrafluoroethylene (PTFE), perfluoroethylene-propylene (PFEP), perfluoroalkoxy alkane (PFA), ethylene tetrafluoroethylene (ETFE), polyvinylidene fluoride (PVDF), chlorinated ethylene tetrafluoroethylene, or another coating treatment that creates a barrier to chemical interaction between the composition and the container.

The valve assembly may comprise a valve, a spring, a dip tube, an actuator, and a dust cap. Various types of valves such as continuous spray valves and metering valves can be used. The metered valve dispenses a metered quantity of formulation with each actuation of the actuator. The metered quantity avoids under-dosing or overdosing that may lead to undesirable side effects. A dust cap is fitted onto the container to shield the contents of the container from the outside environment.

The amount of halobetasol depends upon the purpose for which the composition is to be applied. For example, the dosage and frequency of application can vary depending upon the type and severity of the topical condition.

The following examples represent various embodiments according to the present invention. The examples are given solely for the purpose of illustration and are not to be construed as limitations of the present invention, as many variations thereof are possible without departing from the spirit and scope of the invention.

### EXAMPLES

### Example 1

| Ingredients | Quantity (% w/w) |
|---|---|
| Halobetasol propionate | 0.05 |
| Ethyl alcohol | 52.65 |
| Isopropyl myristate | 7.30 |
| Liquefied petroleum gas | 40.00 |

Procedure:
1. Halobetasol propionate is dissolved into a portion of ethyl alcohol while stirring.
2. Isopropyl myristate is added while stirring into the solution of step 1.
3. The remaining quantity of ethyl alcohol is added into the solution of step 2 and mixed.
4. The solution of step 3 is filled into an aluminum container with an inert liner, and the container is fitted with a valve assembly.
5. Liquefied petroleum gas is charged into the filled container of step 4.

### Example 2

| Ingredients | Quantity (% w/w) |
|---|---|
| Halobetasol propionate | 0.05 |
| Ethyl alcohol | 32.65 |
| Isopropyl myristate | 7.30 |
| Isobutane | 60.00 |

Procedure:
1. Halobetasol propionate is dissolved into a portion of ethyl alcohol while stirring.
2. Isopropyl myristate is added while stirring into the solution of step 1.
3. The remaining quantity of ethyl alcohol is added into the solution of step 2 and mixed.
4. The solution of step 3 is filled into an aluminum container with an inert liner, and the container is fitted with a valve assembly.
5. Isobutane is charged into the filled container of step 4.

### Example 3

| Ingredients | Quantity (% w/w) |
|---|---|
| Halobetasol propionate | 0.05 |
| Ethyl alcohol | 32.65 |
| Isopropyl myristate | 7.30 |
| Butane | 60.00 |

Procedure:
1. Halobetasol propionate is dissolved into a portion of ethyl alcohol while stirring.
2. Isopropyl myristate is added while stirring into the solution of step 1.
3. The remaining quantity of ethyl alcohol is added into the solution of step 2 and mixed.
4. The solution of step 3 is filled into an aluminum container with an inert liner, and the container is fitted with a valve assembly.
5. Butane is charged into the filled container of step 4.

### Example 4

| Ingredients | Quantity (% w/w) |
|---|---|
| Halobetasol propionate | 0.05 |
| Ethyl alcohol | 32.65 |
| Isopropyl myristate | 7.30 |
| Propane | 60.00 |

Procedure:
1. Halobetasol propionate is dissolved into a portion of ethyl alcohol while stirring.
2. Isopropyl myristate is added while stirring into the solution of step 1.
3. The remaining quantity of ethyl alcohol is added into the solution of step 2 and mixed.
4. The solution of step 3 is filled into an aluminum container with an inert liner, and the container is fitted with a valve assembly.
5. Propane is charged into the filled container of step 4.

### Example 5

| Ingredients | Quantity (% w/w) |
|---|---|
| Halobetasol propionate | 0.05 |
| Ethyl alcohol | 52.65 |
| Isopropyl palmitate | 7.30 |
| Liquefied petroleum gas | 40.00 |

Procedure:
1. Halobetasol propionate is dissolved into a portion of ethyl alcohol with stirring.
2. Isopropyl palmitate is added while stirring into the solution of step 1.
3. The remaining quantity of ethyl alcohol is added into the solution of step 2 and mixed.
4. The solution of step 3 is filled into an aluminum container with an inert liner, and the container is fitted with a valve assembly.
5. Liquefied petroleum gas is charged into the filled container of step 4.

### Example 6

| Ingredients | Quantity (% w/w) |
|---|---|
| Halobetasol propionate | 0.05 |
| Ethyl alcohol | 32.65 |
| Isopropyl palmitate | 7.30 |
| Isobutane | 60.00 |

Procedure:
1. Halobetasol propionate is dissolved into a portion of ethyl alcohol while stirring.
2. Isopropyl palmitate is added while stirring into the solution of step 1.
3. The remaining quantity of ethyl alcohol is added into the solution of step 2 and mixed.
4. The solution of step 3 is filled into an aluminum container with an inert liner, and the container is fitted with a valve assembly.
5. Isobutane is charged into the filled container of step 4.

### Example 7

| Ingredients | Quantity (% w/w) |
|---|---|
| Halobetasol propionate | 0.05 |
| Ethyl alcohol | 19.95 |
| Propylene glycol | 30.00 |
| Isobutane | 50.00 |

Procedure:
1. Halobetasol propionate is dissolved into a portion of ethyl alcohol while stirring.
2. Propylene glycol is added while stirring into the solution of step 1.
3. The remaining quantity of ethyl alcohol is added into the solution of step 2 and mixed.
4. The solution of step 3 is filled into an aluminum container with an inert liner, and the container is fitted with a valve assembly.
5. Isobutane is charged into the filled container of step 4.

### Example 8

| Ingredients | Quantity (% w/w) |
|---|---|
| Halobetasol propionate | 0.05 |
| Ethyl alcohol | 19.95 |
| Propylene glycol | 30.00 |
| Liquefied petroleum gas | 50.00 |

Procedure:
1. Halobetasol propionate is dissolved into a portion of ethyl alcohol while stirring.
2. Propylene glycol is added while stirring into the solution of step 1.
3. The remaining quantity of ethyl alcohol is added into the solution of step 2 and mixed.
4. The solution of step 3 is filled into an aluminum container with an inert liner, and the container is fitted with a valve assembly.
5. Liquefied petroleum gas is charged into the filled container of step 4.

### Example 9

| Ingredients | Quantity (% w/w) |
|---|---|
| Halobetasol propionate | 0.05 |
| Ethyl alcohol | 42.65 |
| Isopropyl palmitate | 7.30 |
| Liquefied petroleum gas | 50.00 |

Procedure:
1. Halobetasol propionate was dissolved into a portion of ethyl alcohol while stirring.
2. Isopropyl palmitate was added while stirring into the solution of step 1.
3. The remaining quantity of ethyl alcohol was added into the solution of step 2 and mixed.
4. The solution of step 3 was filled into an aluminum container with an inert liner, and the container was fitted with a valve assembly.
5. Liquefied petroleum gas was charged into the filled container of step 4.

### Example 10

| Ingredients | Quantity (% w/w) |
|---|---|
| Halobetasol propionate | 0.05 |
| Ethyl alcohol | 42.65 |
| Isopropyl palmitate | 7.30 |
| Isobutane | 50.00 |

Procedure:
1. Halobetasol propionate was dissolved into a portion of ethyl alcohol while stirring.
2. Isopropyl palmitate was added while stirring into the solution of step 1.
3. The remaining quantity of ethyl alcohol was added into the solution of step 2 and mixed.
4. The solution of step 3 was filled into an aluminum container with an inert liner, and the container was fitted with a valve assembly.
5. Isobutane was charged into the filled container of step 4.

### Example 11

| Ingredients | Quantity (% w/w) |
|---|---|
| Halobetasol propionate | 0.05 |
| Ethyl alcohol | 20.00 |
| Caprylic and capric triglycerides | 29.95 |
| (Crodamol ™ GTCC-LQ) | |
| Isobutane | 50.00 |

Procedure:
1. Halobetasol propionate was dissolved into a portion of ethyl alcohol while stirring.
2. Crodamol™ GTCC-LQ was added while stirring into the solution of step 1.
3. The remaining quantity of ethyl alcohol was added into the solution of step 2 and mixed.
4. The solution of step 3 was filled into an aluminum container with an inert liner, and the container was fitted with a valve assembly.
5. Isobutane was charged into the filled container of step 4.

### Example 12

| Ingredients | Quantity (% w/w) |
|---|---|
| Halobetasol propionate | 0.05 |
| Ethyl alcohol | 20.00 |
| Caprylic and capric triglycerides | 29.95 |
| (Crodamol ™ GTCC-LQ) | |
| Liquefied petroleum gas | 50.00 |

Procedure:
1. Halobetasol propionate was dissolved into a portion of ethyl alcohol with stirring.
2. Crodamol™ GTCC-LQ was added while stirring into the solution of step 1.
3. The remaining quantity of ethyl alcohol was added into the solution of step 2.
4. The solution of step 3 was filled into an aluminum container with an inert liner, and the container was fitted with a valve assembly.
5. Liquefied petroleum gas was charged into the filled container of step 4.

We claim:

1. A topical spray composition of halobetasol comprising halobetasol, an emollient, a non-aqueous solvent, and a propellant.

2. The topical spray composition of claim 1, wherein the emollient is selected from the group consisting of fatty acid triglycerides, fatty acid esters, polyhydric alcohols, fatty acids, oils, cyclomethicone, hydrogenated lanolin, waxes, lecithin, or mixtures thereof.

3. The topical spray composition of claim 2, wherein the emollient is selected from the group consisting of fatty acid triglycerides, fatty acid esters, and polyhydric alcohols.

4. The topical spray composition of claim 1, wherein the non-aqueous solvent is selected from the group consisting of ethyl alcohol, isopropyl alcohol, propylene glycol, butanediol, pentanediol, hexanediol, triethylene glycol, tetraethylene glycol, dipropylene glycol, dibutylene glycol, glycerin, dimethyl isosorbide, tetrahydro furfuryl alcohol polyethylene glycol ether, N-methyl-2-pyrrolidone, 1-methyl-2-pyrrolidinone, dimethyl sulfoxide, dimethyl acetamide, lactic acid, glycolic acid, methylene chloride, methyl-ethyl-ketone, ethyl acetate, methylene dimethyl ether, and mixtures thereof.

5. The topical spray composition of claim 1, wherein the propellant is selected from the group consisting of propane, butane, isobutane, cyclopropane, 1,1,1,2 tetrafluorethane, 1,1,1,2,3,3,3 heptafluoropropane, 1,1, difluoroethane, 1,1,1,3,3,3 hexafluoropropane, fluorocarbon gases, liquefied petroleum gas, and mixtures thereof.

6. The topical spray composition of claim 1, wherein the composition further comprises solubilizers, permeation enhancers, film-formers, plasticizers, antioxidants, pH-adjusting agents, or mixtures thereof.

7. A dispensing system for administering topical spray composition of halobetasol comprising halobetasol, an emollient, a non-aqueous solvent, and a propellant, wherein the dispensing system comprises a container and a valve assembly.

8. A process for the preparation of a topical spray composition of halobetasol, wherein the process comprises the steps of:
(a) dissolving halobetasol in one portion of a non-aqueous solvent;
(b) mixing an emollient and another portion of the non-aqueous solvent into the solution of step (a);
(c) dispensing the solution of step (b) in a dispensing system; and
(d) charging a propellant in the dispensing system of step (c).

9. A method of treating a topical skin condition by administering a topical spray composition of halobetasol comprising halobetasol, an emollient, a non-aqueous solvent, and a propellant.

10. The method of claim 9, wherein the condition is selected from the group consisting of dermatoses, psoriasis, eczema, rosacea, acne vulgaris, dermatitis, pruritus, seborrhea, skin cancers, inflammation, and combinations thereof.
